(19) United States
(12) Patent Application Publication
(10) Pub. No.: US 2004/0243080 A1
(43) Pub. Date: Dec. 2, 2004

(54) Absorbent pads

(75) Inventor: Samuel Charles Baer, Greenville, SC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: BBA Nonwovens Simpsonville, Inc.

(21) Appl. No.: 10/445,539

(22) Filed: May 27, 2003

(51) Int. Cl. A61F 13/15; A61F 13/20

(52) U.S. Cl. 604/378

(57) ABSTRACT

Hydrophilic nonwoven fabric laminates suitable for use as a liquid permeable layer within absorbent pads are provided. The nonwoven laminates generally include at least two layers: a spunbond layer of continuous filament fibers and a meltblown layer of discontinuous filaments. The hydrophilic laminate may further include either a hydrophilic coating or additive. The resulting absorbent pads provide an advantageous balance of properties, including increased wicking speed and height and improved containment characteristics.

### FIELD OF THE INVENTION

The present invention relates to absorbent pads, and more specifically, to an improved absorbent pad having utility in personal hygiene applications or in food packaging.

### BACKGROUND OF THE INVENTION

It is conventional practice to display meat, poultry, and other food products in individual packages for sale. The individual packages generally include a bottom supporting tray and an outer plastic wrap that covers and typically surrounds the supporting tray. Meat or poultry packaged in this manner can exude liquids over time. These exudates tend to pool in the bottom of the supporting tray. In addition to their detrimental aesthetic impact, foodstuff exudates support the rapid growth of bacteria. Left unchecked, the bacteria can migrate from the exudates back to the food product, resulting in spoilage and discoloration.

Absorbent pads are typically placed between the foodstuff and the supporting tray, to capture exudates and thus extend the shelf-life of the product. Such absorbent pads typically include an intermediate absorbent core disposed between a moisture barrier film in contact with the foodstuff and liquid permeable layer in contact with the supporting tray. The absorbent core stores the liquid exudates over time. The absorbent core is typically formed from a fibrous batting, such as cellulosic fiber, which may further include particles of superabsorbent polymer (“SAP”) to trap greater amounts of exudates. The moisture barrier film protects the foodstuffs from reverse migration by the absorbed exudates. The liquid permeable layer allows the exudates that have pooled at the bottom of the supporting tray to pass through to the absorbent core. The moisture barrier film and the liquid permeable layer further act as a containment means for the absorbent core material. Direct contact between the absorbent core components and the foodstuff contaminates the food and is thus highly undesirable.

The liquid permeable layer has traditionally been formed from perforated film. Unfortunately, perforated films cannot wick fluid pools from the edge of the meat pad to a point where they can be absorbed into the core. Perforated films especially suffer from inadequate fluid transfer properties along the plane parallel to the foodstuff. Further, loose portions of the fibrous batting and/or any superabsorbent particles within the absorbent core can readily pass through perforated films.

More recently, nonwoven fabrics formed from spunbond or carded fiber have been used as liquid permeable layers. Although such fabrics do have improved wicking abilities in comparison to perforated films, there remains room for significant improvement. Spunbond and carded fiber fabrics employed in absorbent pads to date further suffer from potential containment issues. More specifically, the spunbond and carded fiber nonwovens used in absorbent cores tend to be fairly light weight, primarily due to economic considerations. Consequently, minor defects in web formation can readily result in larger than normal interstices between the spunbond or carded fibers, allowing core components to sift out of the absorbent pad. Thus there remains a need for liquid permeable layers with improved wicking characteristics and containment properties.

### SUMMARY OF THE INVENTION

The invention provides liquid permeable layers having a desirable balance of properties, including wicking, containment and strength properties, as well as absorbent pads incorporating the same. The liquid permeable layers of the invention generally include a hydrophilic multilayered nonwoven fabric formed from at least one spunbond or carded layer adhered to a meltblown layer.

The absorbent pads of the invention can be used as components in a variety of absorbent applications, including food packaging applications, personal hygiene pads and disposable diapers. The present absorbent pads are particularly well suited for meat and poultry packaging applications. The spunbond or carded layer provides good abrasion resistance and strength to the liquid permeable layer. The meltblown layer provides improved wicking and containment properties. When used in food packaging, the liquid permeable layer of the absorbent pad wicks liquid exudates into the absorbent core, thereby prolonging the shelf-life of the food. The absorbent pads of the invention are further formed from fabrics having improved containment properties, decreasing the potential for contamination of the foodstuffs by the absorbent core.

In addition to a liquid permeable layer, the absorbent pads of the invention further include a liquid impermeable layer and an absorbent core. More particularly, the absorbent pads of the invention generally include a liquid impermeable first layer defining a first outer surface of the absorbent pad, a liquid permeable second layer defining a second outer surface of the absorbent pad, and an absorbent core disposed between the first and second layers. The liquid permeable second layer is formed from a hydrophilic multilayered nonwoven fabric that includes a first nonwoven ply of spunbond or carded fibers which forms the second outer surface of the absorbent pad and a second nonwoven ply of meltblown fibers disposed adjacent to the first nonwoven ply. In advantageous embodiments, the first nonwoven ply is a spunbond nonwoven formed from randomly arranged substantially continuous filaments having a fineness of about 1 to 5 denier per filament and the meltblown fibers have a diameter of from about 0.5 to 10μ. In such advantageous embodiments, the multilayered nonwoven fabric may further include a composition imparting hydrophilic properties to the second layer.

Liquid permeable layers according to the invention can be readily manufactured according to another aspect of the invention. The liquid permeable layers may be manufactured by forming a multilayered web including at least one outer spunbond or carded fiber layer and at least one meltblown layer. Thereafter, the layers of the resultant liquid permeable layer are subjected to a bonding treatment, such as a thermal bonding treatment, sufficient to introduce a plurality of discrete bonds that provide cohesion to the fabric. A surfactant composition may subsequently be applied to one or both surfaces of the liquid permeable layer to either impart of improve the hydrophilicy of the layer.

Surprisingly, the liquid permeable layers of the invention provide a combination of desirable and yet apparently opposing properties in one fabric. More particularly, the liquid permeable layers of the invention provide both improved liquid transfer characteristics, i.e. wicking, and decreased solid transfer characteristics, i.e. increased containment properties.

### BRIEF DESCRIPTION OF THE DRAWINGS

In the drawings which form a portion of the original disclosure of the invention:

FIG. 1 is an exploded perspective view of an absorbent pad in accordance with the invention;

FIG. 2 is an exploded perspective view of a liquid permeable layer in accordance with the invention;

FIG. 3 is a cross-sectional view of food packaging incorporating an absorbent pad of the invention;

FIG. 4 schematically illustrates one method for forming a liquid permeable layer of the invention; and

FIG. 5 schematically illustrates one method for forming an absorbent pad in accordance with the invention.

### DETAILED DESCRIPTION OF THE INVENTION

The present invention will now be described more thoroughly hereinafter with reference to the accompanying drawings, in which illustrative embodiments of the invention are shown. This invention may, however, be embodied in many different forms and should not be construed as limited to the embodiments set forth herein. Rather, this embodiment is provided so that the disclosure will be thorough and complete, and will convey fully the scope of the invention to those skilled in the art. Like numbers refer to like elements throughout. For purposes of clarity, the scale has been exaggerated.

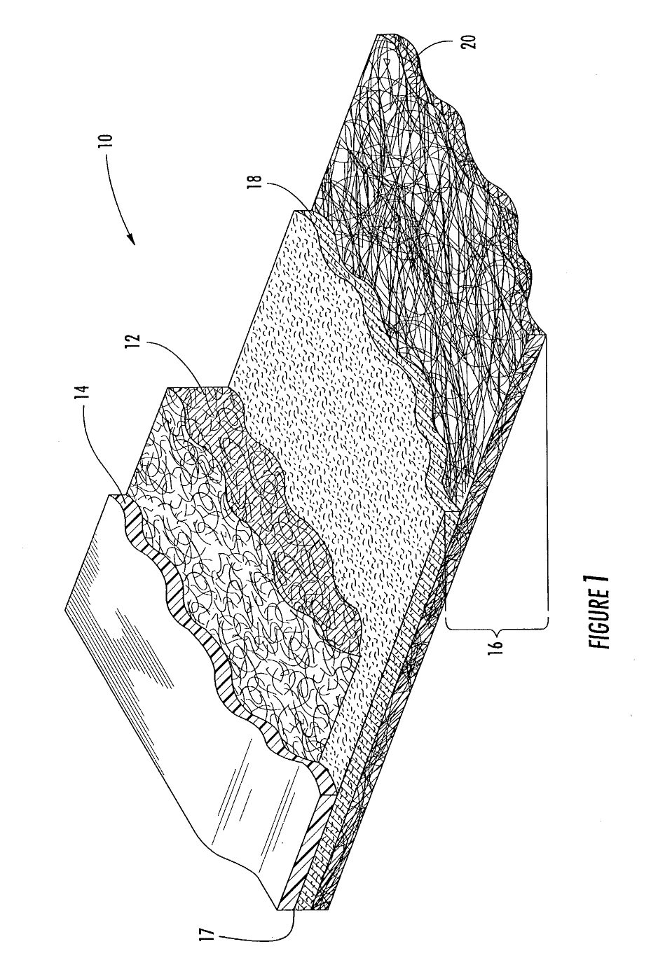

FIG. 1 illustrates an absorbent pad in accordance with the present invention. The absorbent pad is designated generally as 10. In the embodiment provided in FIG. 1, the absorbent pad 10 is a five ply composite that includes an absorbent core 12 disposed between a liquid impermeable layer 14 and a liquid permeable layer 16. The liquid impermeable layer 14 and the liquid permeable layer 16 are bonded together at the periphery of the absorbent pad 10, as indicated at 17, to encapsulate the absorbent core 12 between them. In addition to beneficial liquid transport and containment properties, the absorbent pad 10 has good strength, flexibility and drape and may generally be used to absorb liquids exuded by any source, preferably from foodstuffs and the like.

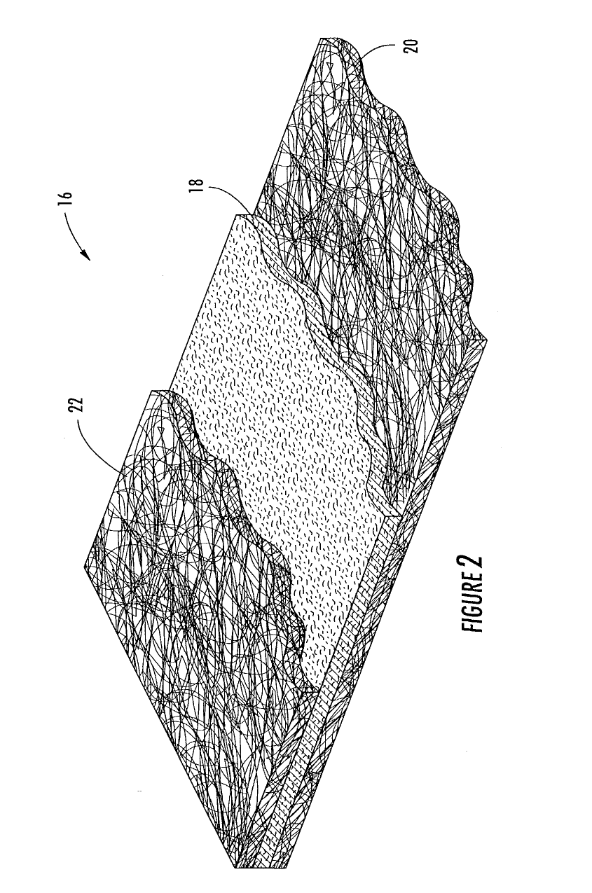

In the embodiment shown in FIG. 1, the liquid permeable layer 16 is a two ply structure formed from a nonwoven ply of meltblown fibers 18 disposed adjacent to and in liquid communication with an outer nonwoven ply of spunbond or carded fiber 20. However, in other embodiments of the invention, such as the embodiment shown in FIG. 2, the liquid permeable layer 16 is a three ply structure formed from a nonwoven ply of meltblown fibers 18 disposed between and in liquid communication with two outer nonwoven plies 20 and 22, each of which may independently be formed from either a spunbond web or a carded fiber web. For the sake of clarity and brevity, the invention will generally be described herein in terms of the three layered liquid permeable film structure illustrated in FIG. 2. Further, although the liquid permeable layer is referred to as containing “layers” or “plies,” these terms are merely used to facilitate discussion concerning the differing compositions which may be present in various regions within the liquid permeable layer thickness. The liquid permeable layers of the present invention, although referred to as being formed from such “layers” or “plies” nevertheless provide unitary structures exhibiting cohesive properties throughout their thickness. Each “layer” or “ply” is also generally in direct liquid communication with its adjacent layer(s).

The nonwoven ply of meltblown fibers 18 can be made from any of a number of known fiber forming polymer compositions. As used herein, the term “composition” is used to refer a chemical preparation containing one or more components. Exemplary polymers from which the meltblown fibers may be formed include polyolefins, such as polyethylene, polypropylene, polybutene, polypentene and copolymers and blends thereof. The terms “polypropylene” and “polyethylene” are used herein in a general sense, and are intended to include various homopolymers, copolymers, terpolymers, and blends thereof. The term “polyethylene” is also intended to include any polyethylene suitable for fiber formation including low density polyethylene, high density polyethylene, and linear low density polyethylene. The meltblown fibers of intermediate ply 18 are preferably formed from polypropylene.

The meltblown fibers forming the nonwoven ply 18 can be of any size known in the art of meltblown webs. The meltblown fibers preferably have an average fiber diameter of up to about 10 microns with very few, if any, of the fibers exceeding 10 microns in diameter. Typically, the diameter of the fibers will range from about 4 to 6 microns. As known in the art, meltblown fibers are typically discontinuous. The nonwoven ply of meltblown fibers 18 is preferably manufactured in accordance with the process described in Buntin et al., U.S. Pat. No. 3,978,185.

The nonwoven ply of meltblown fibers 18 can have a basis weight in the range of about 1.25 to 20 grams per square meter (gsm), advantageously in the range of about 2 to 5 gsm, preferably about 2.5 gsm. Surprisingly, fairly low basis weights of meltblown fiber, such as basis weights ranging from about 2 to 3 gsm, create an effective barrier to the absorbent core components, such as fibrous batting and SAP. Although not wishing to be bound by theory, Applicant hypothesizes that only minimal amounts of meltblown fiber are required to effectively “fill in” any defects present within the outer spunbond or carded fiber web.

In the embodiment illustrated in FIG. 2, the liquid permeable layer 16 includes a single nonwoven ply of meltblown fibers 18. In alternative embodiments, the liquid permeable layer 16 includes more than a single nonwoven ply of meltblown fibers 18. For example, the liquid permeable layer 16 may include two meltblown plies sandwiched between the outer nonwoven plies of spunbond or carded fiber 20 and 22. For embodiments including at least two meltblown plies, the meltblown plies may be the same or may differ. For example, the meltblown plies may differ in composition, average denier, or basis weight.

The spunbond or carded fibers which form the outer nonwoven plies 20 and 22 of the liquid permeable layer can independently be made of any of a number of known fiber forming polymer compositions. Exemplary polymers from which the spunbond or carded fibers may be formed include polyolefins, such as polyethylene, polypropylene, polybutene, polypentene and copolymers and blends thereof. The spunbond or carded fibers of the outer nonwoven plies 20 and 22 of the liquid permeable nonwoven are preferably formed from polypropylene.

Spunbond fibers in accordance with the invention generally range in size from about 2.0 to 4.0 denier per filament, such as from about 2.0 to 3.0. Carded fibers in accordance with the invention generally range from about 2.0 to 4.0 denier per filament, such as from about 2.0 to 3.0. As known in the art, spunbond nonwoven fabrics are formed from continuous filament fiber, while carded nonwoven fabrics are formed from discrete length staple fiber. Any length of staple fiber known in the art may be used in conjunction with the carded nonwoven fabrics of the invention.

In advantageous embodiments, the outer spunbond or carded plies 20 and 22 of the liquid permeable layer have a total basis weight ranging from about 10 to 20 gsm, such as from about 12 to 18 gsm, preferably about 13 gsm. On an individual basis, each of the outer spunbond or carded plies 20 and 22 may range in basis weight from about 5 to 10 gsm, such as from about 6 to 9 gsm, preferably about 6.5 gsm. The basis weights of the outer nonwoven plies of spunbond or carded fiber 20 and 22 may be approximately the same, or may differ. In preferred embodiments of the invention, the outer spunbond or carded fiber nonwoven layers 20 and 22 have approximately the same basis weight.

The outer nonwoven plies of spunbond or carded fiber 20 and 22 may have any construction associated with and/or be produced by any known spunbonding or carded nonwoven process. Exemplary spunbond fabric constructions include area bonded and point bonded spunbond nonwovens. Exemplary carded fiber nonwoven constructions include carded thermal bond nonwovens and through-air-bonded nonwovens.

The liquid permeable layer 16 typically exhibits a basis weight ranging from about 8 to 50 gsm, such as from about 10 to 30 gsm, preferably about 15.5 gsm. The liquid permeable layer 16 further typically includes from about 5 to 95 weight percent meltblown fiber, such as from about 8 to 32 weight percent, preferably about 16 weight percent. The liquid permeable layer 16 may further typically include from about 5 to 95 weight percent spunbond or carded fiber, such as an amount of about 21 to 84 weight percent, preferably about 42 weight percent. When used for food packaging applications, polymers used to form the various component plies 18, 20 and 22 of the liquid permeable layer 16 are preferably food grade polymers.

The liquid permeable layer 16 is preferably hydrophilic. As used herein, the term “hydrophilic” is used in a general sense to refer to materials that have a surface energy such that the polymeric material is wettable by an aqueous medium, i.e., a liquid medium of which water is a major component. Hydrophilicity may be imparted to the liquid permeable layer 16 by (a) applying a topical solution to the liquid permeable layer 16 as a whole and/or one or more of the component plies 18, 20, 22 and/or (b) by incorporating a hydrophilic additive within the resin used to form at least a portion of the fibers within at least one of the component plies 18, 20, 22.

In preferred embodiments of the invention, a topical solution, e.g. a surfactant composition within an aqueous based solvent, is coated onto at least one side of the liquid permeable layer. In advantageous embodiments, the surfactant composition is applied across the full width of at least one side of the liquid permeable layer. Suitable surfactants include nonionic, cationic, anionic, and amphoteric surfactants. Nonionic surfactants may preferably be employed, particularly nonionic surfactants having a molecular weight distribution that is standard or otherwise conventional. For example, any nonionic surfactant known in the art may be included within the surfactant composition. Non-limiting exemplary nonionic surfactants include alkyl phenol ethoxylates, alcohol ethoxylates and fatty oxides. In preferred embodiments of the invention, the nonionic surfactant is alkyl phenol ethoxylate, particularly octyl phenol ethoxylate. Octyl phenol ethoxylate is commercially available as Triton X-100® surfactant from Dow Chemical Co. of Midland, Michigan. The dried surfactant composition may generally be present within the liquid permeable layer 16 in amounts of at least about 0.1 weight percent, based on the weight of the multilayered fabric, such as an amount of about 0.5 to 1.0 weight percent, and preferably about 0.8 weight percent.

As noted above, a hydrophilic additive composition may be included within the resin, i.e. the melt blend, used to form at least a portion of the fibers within at least one of the component plies 18, 20, 22. In preferred embodiments of the invention, the hydrophilic additive composition is included in substantially all of the fiber within the liquid permeable layer 16. In advantageous alternative embodiments of the invention, the hydrophilic additive composition is included in the nonwoven ply of meltblown fibers 18 alone. Any hydrophilic additive composition known in the art to impart hydrophilic character to fiber forming resins may be used to impart hydrophilic character to the liquid permeable layer. Non-limiting exemplary hydrophilic additives include monomer fatty acids, dimer fatty acids, hydroxy phenols, polyethylene glycol, polyvinyl alcohol, polyvinyl formal and mixtures thereof The hydrophilic additive composition may be included within the resin in amounts known in the art to impart hydrophilic characteristics to fiber forming resins.

Liquid permeable layers 16 in accordance with the invention provide a beneficial balance of contradictory properties, i.e. improved liquid transport and diminished solids transport. The nonwoven ply of meltblown fibers 18 provide improved wicking properties, e.g. wicking speed and distance, to the liquid permeable layer 16. Liquid permeable layers in accordance with the invention typically exhibit vertical wicking heights at 60 seconds of greater than about 30 cm, such as greater than about 35 cm, preferably greater than about 38 cm. In contrast, conventional liquid permeable layers have much lower vertical wicking heights at 60 seconds, such as a height of less than 28 cm, particularly about 27 cm. The improved liquid transport properties of the present liquid permeable layers are unusual in light of the fact that meltblown fabrics typically exhibit lower permeablities than spunbond or carded fabrics.

Although providing improved liquid transport properties, i.e. increased wicking speed and height, liquid permeable layers 16 in accordance with the invention further provide diminished solids transport. More particularly, the present liquid permeable layers 16 provide improved containment of the absorbent core material in comparison to the conventional liquid permeable layers within absorbent pads to date. Thus the liquid permeable layers of the invention provide absorbent pads exhibiting a heretofore unknown balance of beneficial properties.

Returning now to FIG. 1, the absorbent pad 10 further includes an absorbent core 12 adjacent to and in liquid communication with the liquid permeable layer 16. The absorbent core 12 may be formed from any highly absorbent material known in the art to store liquids. For example, the absorbent core 12 can be formed from fibrous material, such as cellulosic, synthetic or regenerated fiber. Non-limiting cellulosic fiber materials include wood pulp, paper, cotton and mixtures thereof. The absorbent core 12 may have any thickness known in the art for the particular absorbent application. For example, absorbent cores suitable for food packaging typically range in thickness from about ⅛ to ¼ inches.

In preferred embodiments of the invention, the absorbent core 12 further comprises particles of superabsorbent polymer. As used herein, the term “superabsorbent polymer” includes any substantially water-insoluble polymeric material capable of absorbing large quantities of fluid in relation to its weight. The SAP can be in the form of particulate matter, flakes, fibers and the like. Exemplary particulate forms include granules, pulverized particles, spheres, aggregates and agglomerates. Exemplary SAP include polyacrylamides, polyvinyl alcohol, polyacrylates, various grafted starches, and the like. Superabsorbent polymer materials are commercially available. The SAP may be included within the absorbent core 12 in any effective amount known in the art. At least a portion of the SAP may further be adhesively bonded. Exemplary adhesives that may be used to bond the SAP include thermoplastic, pressure sensitive and cured latex adhesives.

The absorbent core 12 may further be mechanically interconnected to maintain the relative positioning thereof and the integrity of the fibrous batting, as known in the art. In addition, a bacterial agent, such as potassium sorbate, may be added to the absorbent core to retard bacterial growth.

As shown in FIG. 1, the absorbent pad 10 further includes a liquid impermeable layer 14. The liquid impermeable layer 14 is typically composed of a material which is non-reactive to food products, e.g. a food grade polymer. The liquid impermeable layer 14 is typically a flexible barrier film. The barrier film may be any non-porous moisture resistant film known in the art, such as a polyolefin film. In advantageous embodiments, the liquid impermeable layer 14 is a polyethylene film. The liquid impermeable layer 14 may have any thickness known in the art of absorbent pads. For example, the liquid impermeable layer 14 may have a thickness ranging from about 0.00035 to 0.005 inches.

The peripheral edges of the liquid permeable layer 16 and the liquid impermeable layer 14 are preferably sealably secured together as shown at 17 by any suitable means, such as an adhesive seal, a thermal bond seal, and the like to thereby sealably enclose the absorbent core 12 between the liquid impermeable layer 14 and the liquid permeable layer 16.

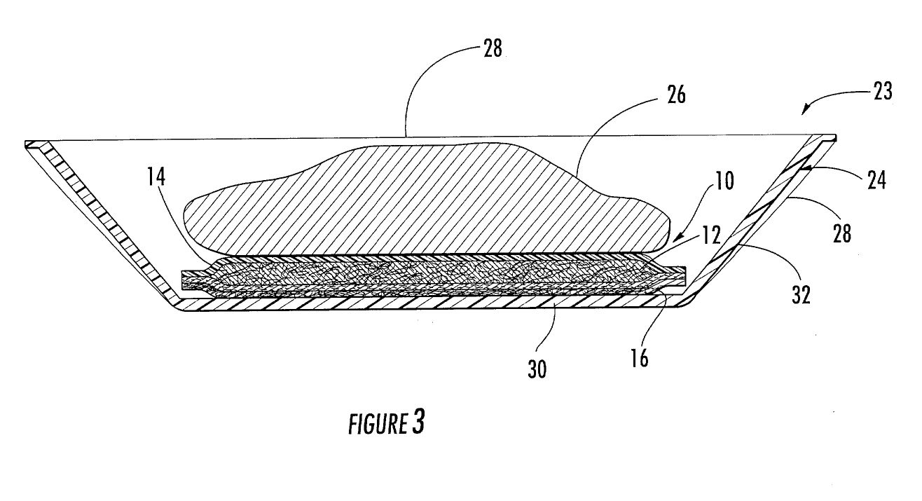

FIG. 3 is a cross-sectional view of food packaging 23 incorporating an absorbent pad formed in accordance with the invention. The food packaging 23 of the invention generally includes a support tray 24, an absorbent pad 10 for trapping liquid exudates emitted by foodstuffs 26 and a plastic film overwrap 28. The support tray 24 may be fabricated from a relatively rigid molded foam plastic material, and generally includes a bottom wall 30 and an upstanding peripheral side wall 32. The bottom wall 30 is typically substantially rectangular in shape. The absorbent pad 10 is sized to overlie substantially the full area of the bottom wall 30 of the support tray 24, and thus has the same general shape as the bottom wall 30. For example, in embodiments in which the bottom wall 30 is rectangular, the absorbent pad 10 is rectangular as well. In use the absorbent pad 10 is positioned upon the bottom wall 30 and the foodstuff 26 is positioned to rest on the absorbent pad 10. A plastic film overwrap 28 may be positioned over the foodstuff and sealed beneath the tray as known in the art, to form the completed food package 23.

In use, the exudates from the foodstuff 26 resting on the absorbent pad 10 will tend to flow downwardly onto the bottom wall 30 of the support tray 24 and beneath the absorbent pad 10. The liquid permeable layer 16 transports the exudates into the absorbent core 12, where they are held out of contact from the foodstuff 26. The liquid impermeable layer 14 prevents the absorbed exudates from directly contacting the foodstuffs 26. The liquid permeable layer 16 provides superior wicking properties in comparison to conventional liquid permeable layers. The liquid permeable layer 16 further exhibits improved containment characteristics. Thus exudates are more readily transferred through the liquid permeable layer 16 and the absorbed exudates and absorbent core materials are subsequently more efficiently trapped within absorbent pad 10.

Although shown in FIG. 3 for use within a tray support, the absorbent cores of the invention may further be used in conjunction with plastic bags, such as those commonly used in poultry packaging.

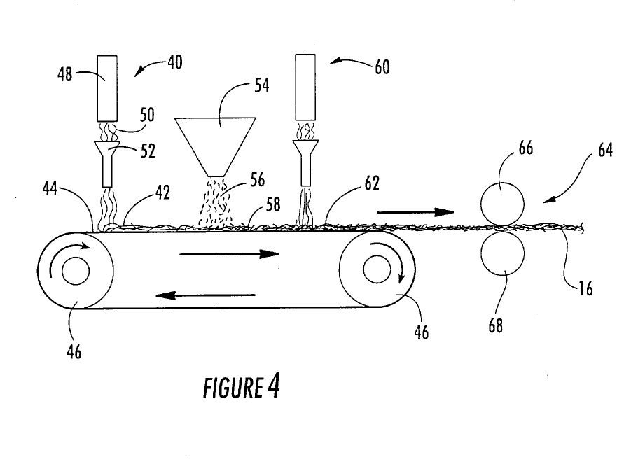

Referring now to FIG. 4, an illustrative process for forming an embodiment of the liquid impermeable layer 16 is illustrated. A conventional spunbonding apparatus 40 forms a first spunbond layer 42 of substantially continuous polymer filaments. Advantageously, the polymer filaments are polypropylene filaments. The first spunbond layer 42 is deposited onto a forming wire or screen 44 which is driven in a longitudinal direction by rolls 46.

The spunbonding process 40 generally involves extruding a polymer through a generally linear die head or spinneret 48 for melt spinning substantially continuous filaments 50, preferably in substantially equally spaced arrays. The substantially continuous filaments 50 are extruded from the spinneret 48 and subsequently quenched by a supply of cooling air. The quenched filaments are directed to an attenuator 52 and deposited onto the forming wire 44.

The initial spunbond layer 42 then moves longitudinally beneath a conventional meltblowing apparatus 54. Meltblowing apparatus 54 deposits a meltblown stream of discontinuous fiber 56 on the surface of the spunbond web 42 to form a spunbond web/meltblown web structure 58. Advantageously, the meltblown stream 56 is formed from polypropylene. Meltblowing processes and apparatus are known to the skilled artisan and are disclosed, for example, in U.S. Pat. No. 3,849,241 to Buntin et al. and U.S. Pat. No. 4,048,364 to Harding et al.

After the meltblown stream of discontinuous fibers 56 is deposited on the surface of the spunbond web 42, the spunbond web/meltblown web structure 58 is next conveyed by forming screen 44 in the longitudinal direction beneath a second conventional spunbonding apparatus 60. The spunbonding apparatus 60 deposits a second spunbond layer of continuous filaments onto the intermediate structure 58 to thereby form a laminate comprising a spunbond web/meltblown web/spunbond web structure 62. Advantageously, the second spunbonded layer is formed from polypropylene.

The three-layer laminate 62 is conveyed longitudinally as shown in FIG. 4 to a conventional thermal fusion station 64 to provide a liquid permeable layer 16. The fusion station 64 is constructed in a conventional manner as known to the skilled artisan, and advantageously includes cooperating calender rolls 66 and 68, which may include at least one point roll, helical roll, and the like. Preferably, the layers are bonded together to provide a multiplicity of thermal bonds distributed throughout the laminate fabric. Bonding conditions, including the temperature and pressure of the bonding rolls, are known in the art for differing polymers. For composites comprising a polypropylene spunbond web/polypropylene meltblown web/polypropylene spunbond web, the embossing rolls are preferably heated to a temperature between about 120° C. and about 130° C. The laminate is fed through the embossing rolls at a speed of about 3 to 300 meters per minute, such as a speed between about 5 and 150 meters per minute.

Although a thermal fusion station in the form of bonding rolls is illustrated in FIG. 4, other thermal treating stations such an ultrasonic, microwave or other RF treatment zones which are capable of bonding the fabric can be substituted for the bonding rolls of FIG. 4. Such conventional heating stations are known to those skilled in the art and are capable of effecting substantial thermal fusion of the nonwoven webs. In addition, other bonding techniques known in the art can be used, such as hydroentanglement of the fibers, needling, and the like. It is also possible to achieve bonding through the use of an appropriate bonding agent as is known in the art, singly or in combination with thermal fusion.

The resultant liquid permeable layer 16 exits the thermal fusion station and is wound up by conventional means on a roll.

The method illustrated in FIG. 4 is susceptible to numerous variations. For example, although the schematic illustration of FIG. 4 has been described as forming a spunbond web directly during an in-line continuous process, it will be apparent that the spunbond webs can be preformed and supplied as rolls of preformed webs. Similarly, although the meltblown web is shown as being formed directly on the spunbond web, and the spunbond web thereon, meltblown webs and spunbond webs can both be preformed and such preformed webs can be combined to form the liquid permeable layer. Alternatively, preformed spunbond and meltblown webs can be passed through heating rolls for further consolidation and thereafter a spunbond layer may be extruded onto the surface of the intermediate structure. Similarly, the three-layer laminate can be formed and stored prior to bonding.

One or more topical treatments, i.e. coatings, may be applied to the liquid permeable layer 16, either as it exits the nonwoven apparatus or in a separate coating process. For example, a surfactant composition may be applied to the liquid permeable layer 16 to impart hydrophilic properties thereto. A typical surfactant composition includes an effective amount of surfactant dispersed in water. In preferred embodiments of the invention, the surfactant composition may include surfactant in an amount ranging from about 10 to 30 weight percent, particularly about 20 weight percent. In alternative embodiments, the surfactant composition may include up to about 100 weight percent surfactant. Any suitable coating method known in the art may be used to apply the topical treatment to the liquid permeable layer 16. Non-limiting exemplary coating methods include foaming, spraying, dipping, gravure coating and the like. The coating compositions are dried subsequent to their application, as further known in the art. The dried coated liquid permeable layer may then be taken up on a roll and stored.

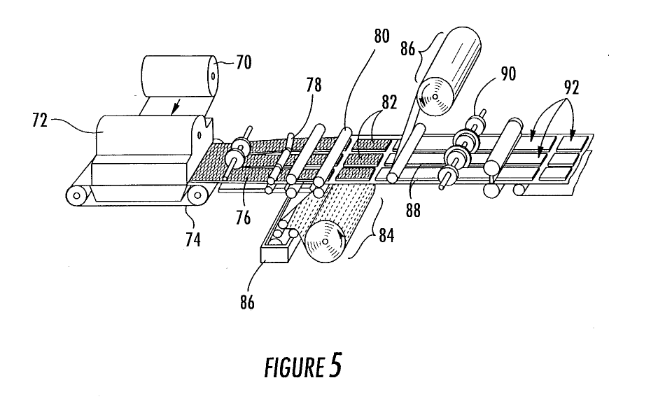

FIG. 5 illustrates one method by which absorbent pads in accordance with the invention may be produced. Generally, the instant absorbent pads are produced by forming individualized absorbent cores across a wide web, arranging the liquid impermeable and liquid permeable layers into position across the wide web, bonding the peripheral edges of the liquid permeable layer to the liquid impermeable layer to encapsulate the individualized absorbent cores, and separating the bonded wide rolls into discrete absorbent pads.

As shown in FIG. 5, a roll of suitable absorbent core material 70 is advanced into a shredding apparatus 72, which reduces the absorbent core material 70 into a fiberized mass, which is then deposited onto a forming screen 74. The fiberized absorbent material is then slit into longitudinal strips 76, which are laterally separated by rolls 78. The strips are then cut transversely 80 to form individualized absorbent cores 82. A roll of liquid impermeable film 84 is fed through an adhesive applicator 86 and then to a position to underlie the individualized absorbent cores 82. The adhesive is preferably applied in a discontinuous pattern to allow the resulting absorbent pad to flex freely. The liquid permeable layer 86 is then brought onto the advancing absorbent pad components and a heat seal between the liquid permeable and liquid impermeable layers is formed in the land areas 88 around the periphery of the individualized absorbent cores. The web of bonded absorbent pads then passes through a slitter 90 to separate the bonded pads into narrow lanes, and the narrow lanes are cut into proper lengths, resulting in the finished absorbent pads 92.

The liquid permeable layers of the invention provide absorbent pads exhibiting a heretofore unknown balance of beneficial properties. More particularly, the liquid permeable layers of the invention provide increased wicking speed and height while further exhibiting improved containment of the absorbent core material in comparison to comparable conventional liquid permeable layers, i.e. liquid permeable layers without a nonwoven ply of meltblown fibers.

The invention will be further illustrated by the following non-limiting example.

### EXAMPLES

### Comparative Example 1

A spunbond web was prepared from polypropylene available from BBA Nonwovens under the trade designation FPNTN 639. The filaments in the first spunbond layer had a denier per filament of about 2 to 3, and the spunbonded web of substantially continuous polypropylene filaments had a basis weight of about 15.3 gsm. A hydrophilic coating was then applied to the sample by immersing the spunbond web in a surfactant composition and subsequently drying the coated web. The surfactant composition contained 20% octyl phenol ethoxylate in water. Octyl phenol ethoxylate is commercially available as Tritong X-100 from Dow Chemical Co. of Midland, Michigan. The dried spunbond layer contained about 0.8 weight percent surfactant.

### EXAMPLE 1

A spunbond web was prepared from the polypropylene used to form Comparative Example 1. The filaments in the spunbond layer had a denier per filament of about 2 to 3, and the spunbonded web of substantially continuous polypropylene filaments had a basis weight of about 15.3 gsm. A meltblown web was prepared by meltblowing polypropylene available from Montell under the trade designation H442H to give a fibrous web having a basis weight of about 2 gsm onto the surface of the spunbonded web. The spunbond and meltblown webs were bonded together by passing the sample through the nip of a cooperating pair of textured and smooth embossing rolls.

A hydrophilic coating was then applied to the sample by submersing the spunbond/meltblown laminate in the surfactant composition applied to Comparative Example 1 and subsequently drying the coated laminate. The dried laminate contained about 0.8 weight percent surfactant.

The samples were tested for vertical wicking to determine the speed and distance of liquid absorption. A test specimen approximately 6 inches in length by about 1 inch wide was cut out of the middle of the sample webs in the extrusion direction and subsequently erected vertically. The samples were dipped into an aqueous bath to a ½ cm liquid depth. Wicking height as a function of time was then ascertained for each sample. The results set forth in Table 1 demonstrate the beneficial wicking properties provided by the liquid permeable layers of the invention.

TABLE 1
Sample Wicking Properties

| Sample | Vertical Wicking Height |
|---|---|
| ID | at 60 Seconds |
| Comp. Ex. 1 | 27.1 cm |
| Ex. 1 | 38.3 cm |

As shown in Table 1, liquid permeable layers in accordance with the invention are superior in both wicking speed and distance in comparison to conventional liquid permeable layers.

The foregoing examples are illustrative of the present invention and are not to be construed as limiting thereof. The invention is defined by the following claims, with equivalents of the claims to be included therein.

That which is claimed:

1. An absorbent pad for trapping liquids, said absorbent pad comprising
a liquid impermeable first layer defining a first outer surface of said absorbent pad;
a liquid permeable second layer defining a second outer surface of said absorbent pad; and
an absorbent core disposed between said first and second layers,
said liquid permeable second layer comprising a hydrophilic multilayered nonwoven fabric including a first nonwoven ply of spunbond or carded fibers forming said second outer surface of said absorbent pad and a second nonwoven ply of meltblown fibers disposed adjacent to said first nonwoven ply.

2. An absorbent pad according to claim 1, wherein said multilayered fabric includes a composition to impart hydrophilic properties to the second layer.

3. An absorbent pad according to claim 2, wherein said composition is present in the second nonwoven ply of meltblown fibers.

4. An absorbent pad according to claim 2, wherein said composition comprises at least one nonionic surfactant selected from the group consisting of alkyl phenol ethoxylate, alcohol ethoxylates and fatty oxides.

5. An absorbent pad according to claim 2, wherein said composition comprises a hydrophilic melt additive incorporated into at least some of the fibers of said second layer.

6. An absorbent pad according to claim 1, including a third nonwoven ply of spunbond or carded fibers disposed adjacent to said meltblown second ply and defining an inner surface of said second layer, said inner surface being positioned in contact with said absorbent core.

7. An absorbent pad according to claim 6, wherein said hydrophilic multilayered nonwoven fabric comprises a spunbond-meltblown-spunbond laminate.

8. An absorbent pad according to claim 6, wherein said multilayered fabric includes a hydrophilic coating in an amount of at least about 0.1 weight percent, based on the weight of the multilayered fabric.

9. An absorbent pad according to claim 1, wherein said liquid impermeable first layer comprises a liquid impermeable polymer film, said absorbent core includes superabsorbent polymer particles, and said liquid permeable second layer comprises a spunbond-meltblown or spunbond-meltblown-spunbond laminate including a surfactant coating.

10. An absorbent pad according to claim 1, wherein said absorbent core further comprises an adhesive bonding at least a portion of said superabsorbent polymer particles together, said adhesive being selected from the group consisting of thermoplastic adhesives, pressure sensitive adhesives and cured latex adhesives.

11. An absorbent pad according to claim 9, in the form of a meat pad, and wherein said first and second layers are formed from food-grade polymers.

12. An absorbent pad according to claim 9, in the form of a personal hygiene pad.

13. An absorbent pad according to claim 9, in which said absorbent pad is a component of a disposable diaper.

14. An absorbent pad for trapping liquids, said absorbent pad comprising
a first layer comprising a liquid impermeable polymer film defining a first outer surface of the absorbent pad;
a liquid permeable second layer defining a second outer surface of the absorbent pad; and
an absorbent core disposed between said first and second layers;
said liquid permeable second layer comprising a hydrophilic multilayered nonwoven fabric including a spunbond nonwoven first ply of randomly arranged substantially continuous filaments of 1 to 5 denier per filament forming said second outer surface of said absorbent pad and a second nonwoven ply of meltblown fibers disposed adjacent to said spunbond ply, said meltblown fibers having a diameter of from 0.5 to 10 μ, and said multilayered nonwoven fabric including a composition imparting hydrophilic properties to said second layer.

15. An absorbent pad according to claim 14, wherein said multilayered fabric includes a coating of a surfactant imparting said hydrophilic properties, said surfactant coating being present in an amount of at least about 0.1 weight percent, based on the weight of the multilayered fabric.

16. An absorbent pad according to claim 14, wherein said multilayered fabric includes a hydrophilic melt additive incorporated into at least some of the fibers of said second layer.

17. An absorbent pad according to claim 16, wherein said hydrophilic melt additive comprises at least one additive selected from the group consisting of monomer fatty acids, dimer fatty acids, hydroxy phenols, polyethylene glycol, polyvinyl alcohol, and polyvinyl formal.

18. An absorbent pad according to claim 14, wherein said spunbond nonwoven first ply is formed from continuous filaments comprising at least one polyolefin selected from the group consisting of polyethylene, polypropylene, polybutene, polypentene and copolymers thereof.

19. An absorbent pad according to claim 18, wherein said polyolefin is polypropylene.

20. An absorbent pad according to claim 18, wherein said meltblown nonwoven second ply is formed from fiber comprising at least one polyolefin selected from the group consisting of polyethylene, polypropylene, polybutene, polypentene and copolymers thereof.

21. An absorbent pad according to claim 20, wherein said polyolefin is polypropylene.

22. An absorbent pad according to claim 14, including a spunbond nonwoven third ply of randomly arranged substantially continuous filaments disposed adjacent to said meltblown second ply and defining an inner surface of the second layer, said inner surface being in contact with said absorbent core.

23. An absorbent pad according to claim 22, wherein said filaments of said spunbond first and third plies are formed from at least one polyolefin selected from the group consisting of polyethylene, polypropylene, polybutene, polypentene and copolymers thereof.

24. An absorbent pad according to claim 23, wherein said polyolefin is polypropylene.

25. An absorbent pad according to claim 14, wherein said second layer has a basis weight ranging from about 8 to 50 gsm.

26. An absorbent pad according to claim 14, wherein said second layer exhibits a vertical wicking height at 60 seconds of greater than about 30 cm.

27. An absorbent pad according to claim 14, wherein said absorbent core comprises at least one cellulo sic material selected from the group consisting of wood pulp, paper and cotton.

28. An absorbent pad according to claim 27, wherein said absorbent core further comprises particles of superabsorbent polymer.

29. An absorbent pad according to claim 28, wherein said absorbent core further comprises an adhesive bonding at least a portion of said superabsorbent polymer particles together, said adhesive being selected from the group consisting of thermoplastic adhesives, pressure sensitives and cured latex adhesives.

30. An absorbent pad according to claim 14, wherein said first layer comprises a hydrophobic polyolefin film.

31. An absorbent pad according to claim 14 wherein said meltblown nonwoven ply is present within said mutlilayered fabric in an amount ranging from about 5 to 95 weight percent of the multilayered fabric.

32. An absorbent pad according to claim 28, wherein said first and second spunbond nonwoven plies each have a basis weight of about 6.5 gsm and said meltblown nonwoven ply has a basis weight of about 2.5 gsm.

33. An absorbent pad according to claim 28, wherein said composition comprises alkyl phenol ethoxylate, present in an amount of about 0.8 weight percent, based on the weight of the mutlilayered fabric.

34. Foodstuff packaging comprising
a support tray;
an absorbent pad for trapping liquid exudates emitted by foodstuffs, said absorbent pad comprising
a liquid impermeable first layer defining a first outer surface of the absorbent pad;
a liquid permeable second layer defining a second outer surface of the absorbent pad; and
an absorbent core disposed between said first and second layers,
said liquid permeable second layer comprising a hydrophilic multilayered nonwoven fabric including a first nonwoven ply of spunbond or carded fibers forming said second outer surface of the pad and a second nonwoven ply of meltblown fibers disposed adjacent to said spunbond ply; and
said foodstuff packaging also including a plastic film overwrap.

35. Foodstuff packaging according to claim 34, wherein said second layer further comprises a second spunbond nonwoven ply disposed between said absorbent core and said meltblown nonwoven ply.

36. Foodstuff packaging according to claim 34, wherein said spunbond nonwoven ply and said meltblown nonwoven ply independently comprise at least one olefin selected from the group consisting of polyethylene, polypropylene, polybutene, polypentene and copolymers thereof.

37. Foodstuff packaging according to claim 34, wherein said spunbond nonwoven ply and said meltblown nonwoven ply are formed from polypropylene.

38. A hydrophilic multilayered fabric comprising
a meltblown nonwoven ply exhibiting a first permeability disposed between two plies of spunbond nonwoven, each of which exhibit a higher permeability than said meltblown nonwoven ply and each of which further defines first and second surfaces,
both of said first surfaces of said spunbond nonwoven plies contacting said meltblown nonwoven ply, and
at least one of said second surfaces of said spunbond nonwoven plies further comprising a hydrophilic composition present across substantially the entire second surface,
said hydrophilic composition consisting essentially of at least one surfactant selected from the group consisting of alkyl phenol ethoxylate, alcohol ethoxylates and fatty oxides.

39. A hydrophilic multilayered fabric according to claim 38, wherein said surfactant is alkyl phenol ethoxylate, and said alkyl phenol ethoxylate is octyl phenol ethoxylate.

40. A hydrophilic multilayered fabric according to claim 38, wherein said surfactant is present in an amount of about 0.8 weight percent, based on the weight of the multilayered fabric.

41. A hydrophilic multilayered fabric according to claim 38 wherein said spunbond nonwoven plies and said meltblown nonwoven ply independently comprise at least one polyolefin selected from the group consisting of polyethylene, polypropylene, polybutene, polypentene and copolymers thereof.

42. A hydrophilic multilayered fabric according to claim 38, wherein said spunbond nonwoven plies and said meltblown nonwoven ply are formed from polypropylene.

43. A hydrophilic multilayered fabric comprising
a meltblown nonwoven ply exhibiting a first permeability disposed between two plies of spunbond nonwoven, each spunbond nonwoven ply exhibiting a higher permeability than said meltblown nonwoven ply, said meltblown and spunbond nonwoven plies formed of monocomponent fiber comprising a blend of polyolefin polymer and hydrophilic melt additive.

44. A hydrophilic multilayered fabric according to claim 43, wherein said hydrophilic melt additive comprises at least one additive selected from the group consisting of monomer fatty acids, dimer fatty acids, hydroxy phenols, polyethylene glycol, fluorohydrocarbons, polyvinyl alcohol, and polyvinyl formal.

45. A method for producing an absorbent pad comprising
(a) directing a plurality of substantially continuous filaments onto a collection surface to form a first spunbond web;
(b) directing a plurality of substantially discontinuous filaments onto the first spunbond web to form a meltblown web;
(c) directing a plurality of substantially continuous filaments onto the meltblown web to form a second spunbond web;
(d) bonding the first spunbond web, the meltblown web and the second spunbond web, thereby forming a multilayered fabric;
(e) arranging the multilayered fabric, an absorbent core, and a barrier film in opposing face-to-face relationship and
(f) bonding the multilayered fabric, absorbent core and barrier film to form an absorbent pad for trapping liquid exudates emitted by foodstuffs.

46. A method for producing an absorbent pad according to claim 45, further comprising the step of applying a surfactant composition to the surface of the multilayered fabric.
